(19) United States
(12) Patent Application Publication
(10) Pub. No.: US 2006/0036134 A1
(43) Pub. Date: Feb. 16, 2006

(54) Telemedicine system

(75) Inventors: Lionel Tarassenko, Oxford (GB); Clive Richard Peggram, Oxford (GB); Paul Michael Hayton, Oxford (GB); Oliver George Gibson, Oxford (GB); Alastair William George, Oxford (GB); Jeremy Stuart Wheeler, Maidenhead (GB)

Correspondence Address: NIXON & VANDERHYE, PC, 901 NORTH GLEBE ROAD, 11TH FLOOR, ARLINGTON, VA, 22203, US

(73) Assignee: e-SAN LIMITED, Oxford (GB)

(21) Appl. No.: 10/528,365

(22) PCT Filed: Sep. 18, 2003
(86) PCT No.: PCT/GB03/04029

§ 371 Date: Jul. 19, 2005

(30) Foreign Application Priority Data
Sep. 18, 2002 (GB) 0221713.1

(51) Int. Cl. G06F 15/16; A61B 5/00

(52) U.S. Cl. 600/300; 709/219

(57) ABSTRACT

A telemedicine system for monitoring chronic conditions such as asthma or diabetes includes an electronic measurement device such as an electronic peak expiratory flow meter or an electronic blood glucose meter, connected to a GPRS cellular telephone. The cellular telephone automatically receives, formats and transmits the data on acquisition by the medical device to a remote server. The server may acknowledge the data and make the data available to a clinician. The server may also analyse the data and provide automatic alerts to the patient and/or clinician in the event of the data causing concern. The formatting and transmission of the data from the telephone to the server occurs in real time as the measurements are taken and is invisible to the patient.

This invention relates to a telemedicine system, and in particular to a system with improved operability, thus making it particularly suitable for home health monitoring.

There are a number of chronic medical conditions in which the sufferers (or patients) are required to measure regularly some physiological parameter which characterises their condition, and to record those values. Typically such patients attend regular clinics where a clinician can review the recorded values and assess the state of health of the patient. For example, it is generally accepted that part of the effective treatment of patients suffering from asthma is the regular monitoring of their condition. In particular, daily self-measurement of lung function by patients enables clinicians to assess the severity of the illness and allows the treatment (for instance the dosage of drugs such as steroids) to be tailored to the patient's needs. Commonly, measurement of lung function is by taking peak expiratory flow readings using a Wright's peak flow meter. Patients record measurements twice daily and enter them on a peak flow graph in a patient diary. However, this system of recording depends not only on the patients remembering to note down the correct figures, but also on them entering the data accurately on the graph. At the clinic there is no way that the clinician can be entirely sure that the figure and the corresponding entry on the graph are an accurate representation of the peak flow at the time. The results are also viewed retrospectively by the clinician, who looks for trends since the last visit to the asthma clinic, and so the figures provide little information with regard to the patient's condition at that particular time, and they have limited predictive value.

Type I diabetes is another chronic condition which can be treated or managed using home monitoring. Type I diabetes is treated with insulin (by injection several times a day) and by eating a healthy diet. However, Type I diabetics need to monitor their blood glucose levels regularly. This typically requires a small blood sample to be obtained by pricking the skin, usually on a finger, and placing the sample on a test strip which is read by an electronic glucose meter. Self-monitoring in this way helps to detect when blood sugar levels may be too low, in which case sugar must be taken (for example a sweet drink or meal), or when the blood sugar levels may become too high (for instance at times of illness). Patients typically attend a diabetes clinic every three months or so for blood tests, recordal of height and weight and blood pressure and other checks, such as eye checks for retinopathy. However, with some patients adherence to the management program (of making regular blood glucose readings) is poor and this increases the risk of developing long-term complications. For instance, readings are often missed, in which case patients sometimes fabricate them, or they may be adjusted when recording them in a patient diary. Better adherence to the management program can decrease the occurrence of long-term diabetic complications.

To overcome some of the problems of manual recordal in a patient diary, various technologically-based recordal systems have been proposed. Typically such proposals have involved the use of an electronic physiological data acquisition unit (such as an electronic glucose meter or electronic peak flow meter as above) whose measurements are downloaded onto a data storage device. The stored data may be reviewed at the regular clinics, or in some telemedicine proposals the data may be transferred to a personal computer and sent to a clinic or clinician via the internet. However, the process of downloading the data and transmitting it to the clinician via the internet requires a familiarity with computer systems which not all patients have or desire to attain. Further, it is time-consuming and often troublesome to obtain a connection via the internet. The system is also problematic if the patient is not at home. So the use of this technology has tended to degrade compliance with self-monitoring techniques rather than improve it. Further, none of these systems have proved useful in practice, because a clinician typically looks after hundreds of patients.

It is an object of the present invention to provide an improved telemedicine system, in particular in which the operability is improved so that it enhances the adherence to self-monitoring by patients.

The present invention provides a telemedicine system in which the physiological data is acquired and transmitted to a remote server automatically upon the readings being taken, without the intervention of the patient. In more detail, the present invention provides a telemedicine system comprising a patient-based physiological data acquisition and transmittal device connectable via a wireless network to transmit physiological data to a remote server, wherein the patient-based measurement and data transmittal device comprises:

an electronic physiological data acquisition unit for measuring one or more physiological parameters of a patient to acquire and output data representing the parameter;

a wireless transmitter which upon receiving the output data from the data acquisition unit automatically transmits the output data via the wireless network to the remote server.

Thus preferably the wireless transmitter is adapted to receive automatically the output data from the physiological data acquisition unit on data acquisition thereby, and thereupon automatically to transmit the output data immediately in real time to the remote server. Preferably the wireless transmitter is adapted to establish a connection to the wireless network automatically when it is switched on and to maintain the connection while switched on. Thus the patient is not required to download the data, this is automatic and immediate upon data acquisition. Further, the transmittal of the data is also automatic, again, without bothering the patient. All the patient has to do is switch the device on, take the reading (at which point the readings are automatically sent to the remote server) and switch the device off.

The wireless network may be a packet-switched network, preferably public, such as the GPRS, 3G, PDC-P or EDGE network.

The wireless transmitter may be a cellular telephone or personal digital assistant (PDA) with cellular telephony capability, currently known as a smart phone. A software application may be provided on the cellular telephone/PDA to interface with the physiological data acquisition unit and to control data transmission to the remote server. Thus the patient can switch on the cellular telephone/PDA, select an icon representing the software application, after which the cellular telephone/PDA automatically interfaces with the data acquisition unit and transmits the data via the wireless network to the remote server. The device may be adapted to check the acquired data for compliance with pre-set conditions, such as concerning the quality or completeness of the readings or the condition of the patient. The data may be displayed on the device so that the patient can see that the readings are complete and assess their condition themselves to some extent. However, the automatic transmittal of the data to the remote server means that the patient cannot self-edit the data.

In the event of a network connection being unavailable, the device stores the data and may automatically re-transmit it later when a connection becomes available.

Preferably the remote server immediately processes the data on reception to check the condition of the patient. It may respond with an acknowledgement of the data, and also perhaps with a message related to the patient's condition (for instance to change the treatment regime or to attend a clinic or to seek emergency medical assistance). The remote server also preferably formats the data for delivery and display to a clinician. Thus a clinician may access the data, for instance by viewing it as a web page via the internet or some other network, and the clinician may also send messages to the patient via the network. The remote server may comprise a data analyser for identifying trends in the data, and a message generator for generating automatically messages to be output to at least one of the patient and clinician. Thus automated responses based on the data and giving useful feedback, and optionally advice, to the patient can be sent immediately.

The fact that the server can automatically analyse the data and alert the relevant clinician means that a closed loop including the clinician is produced in the patient management process.

The wireless transmitter may be in the form of a cellular telephone/PDA separate from the physiological data acquisition unit such as an electronic flow meter, electronic blood glucose meter, blood pressure monitor or heart rate monitor, the two units being connectable, for instance by a cable or short range wireless link such as Bluetooth. Alternatively, the wireless transmitter function may be integrated into the physiological data acquisition unit.

The data sent from the wireless transmitter is preferably time stamped with reference to a secure clock which may be provided in the patient-based physiological data acquisition and transmittal device, and the data sent from the wireless transmitter may be digitally signed. Preferably a secure data store is provided in the patient-based physiological data acquisition and transmittal device.

The data sent from the wireless transmitter may comprise the location of the wireless transmitter and the the information sent from the server to the patient-based physiological data acquisition and transmittal device for display thereon may then be adapted depending on the location of the wireless transmitter.

The information sent from the server to the patient-based physiological data acquisition and transmittal device for display thereon may initiate interaction with the patient, for instance by comprising questions for the patient to answer, and can be adapted depending on the value of the physiological parameter measured by the electronic physiological data acquisition unit.

In one embodiment the electronic physiological data acquisition unit is connectable to the wireless transmitter by a connection comprising a data head including an interface, and advantageously the secure clock for time stamping the data and the secure memory for storing the data.

Another aspect of the invention provides a telemedicine system which incorporates handset delivery of advice relating to changes in medication necessary to control a respiratory condition including asthma. The handset may comprise a graphical device indicating the state of an asthmatic condition relative to an alert level, and the medication advice may be based on readings analysed by software at the server and/or handset.

Yet another aspect of the invention provides a telemedicine system which incorporates handset delivery of geographically local information relevant to the patient condition from a central server, such information being derived from knowledge of the geographic location of the wireless handset and being adapted based on measurement of the patient condition by the telemedicine system.

The local information may comprise local air quality information and weather conditions relevant to patients with respiratory diseases.

The invention will be further described by way of example with reference to the accompanying drawings in which:

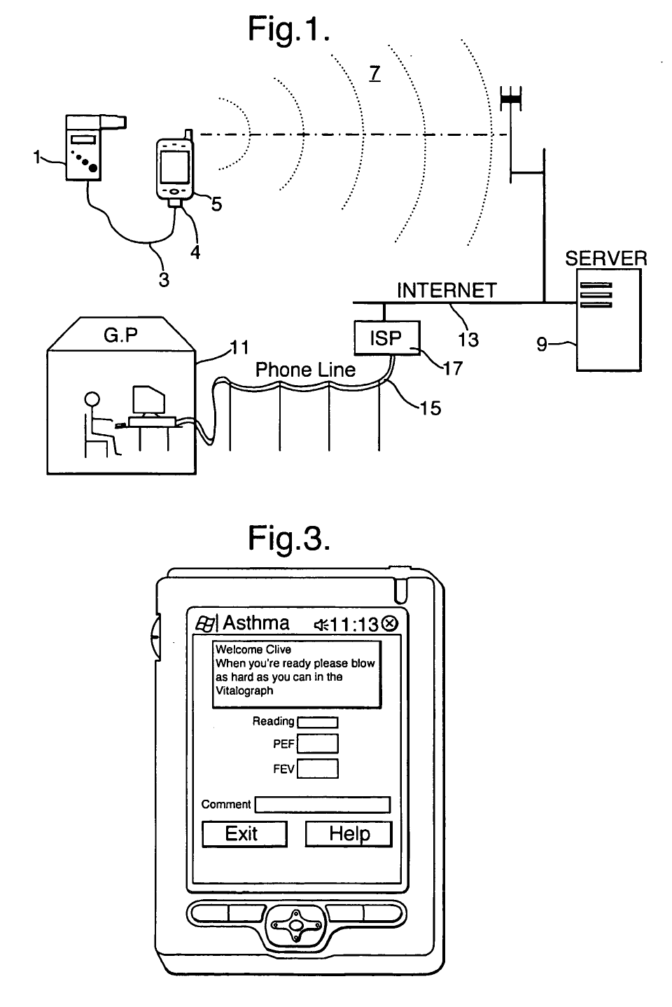

FIG. 1 is a schematic illustration of a first embodiment of the invention;

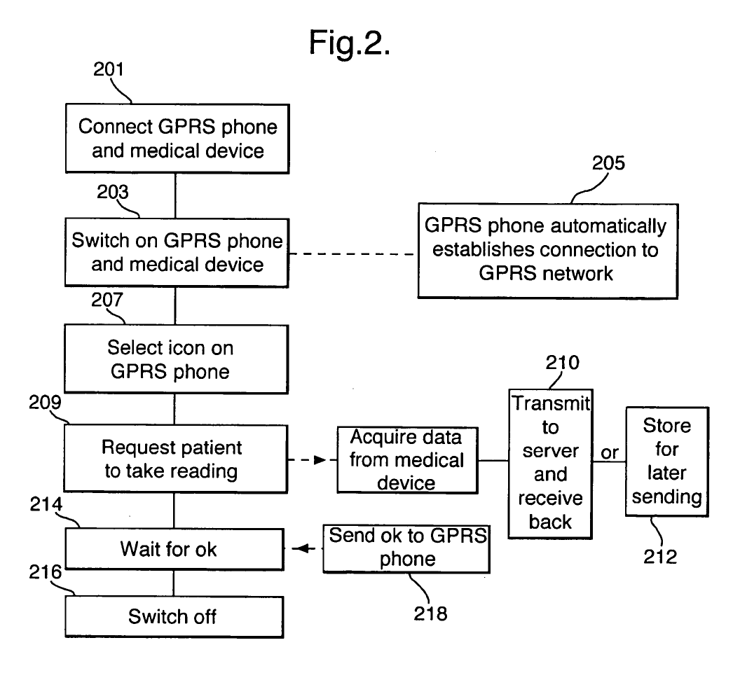

FIG. 2 is a flow diagram showing the operation of the device in one embodiment of the invention;

FIG. 3 illustrates a screen display from the first embodiment of the invention;

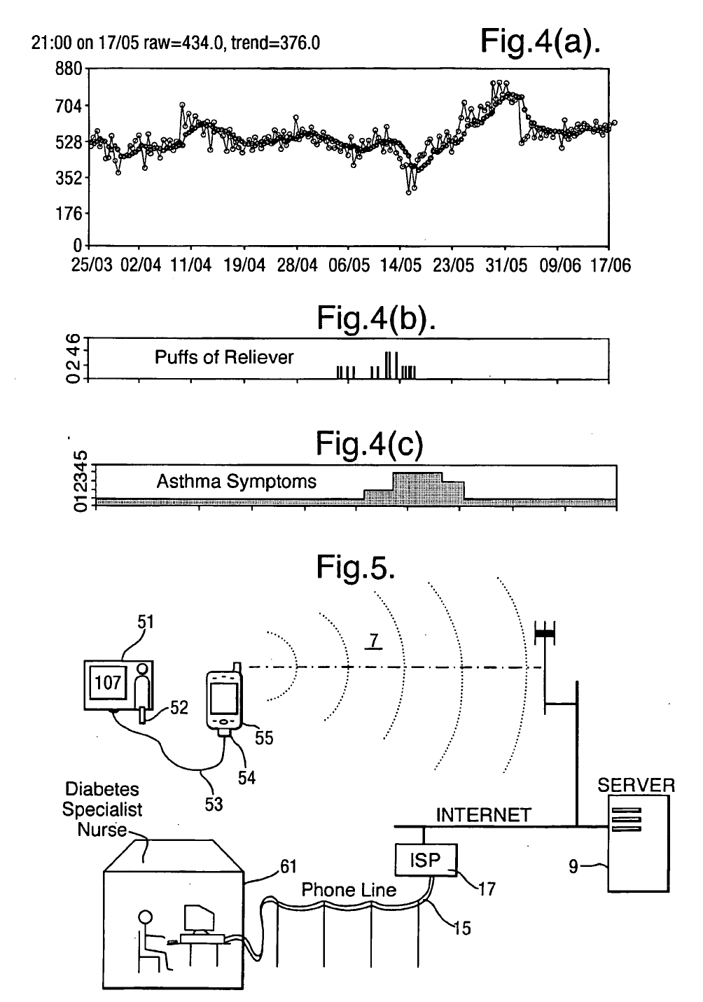

FIG. 4 is a plot of data obtained using an embodiment of FIG. 1;

FIG. 5 is a schematic illustration of a second embodiment of the invention;

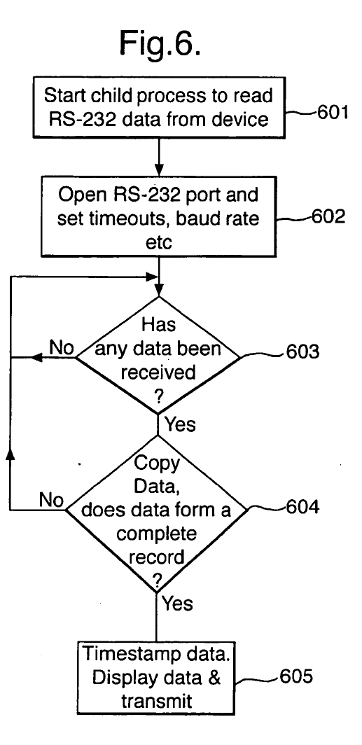

FIG. 6 is a flow diagram of the operation of part of an embodiment of the invention;

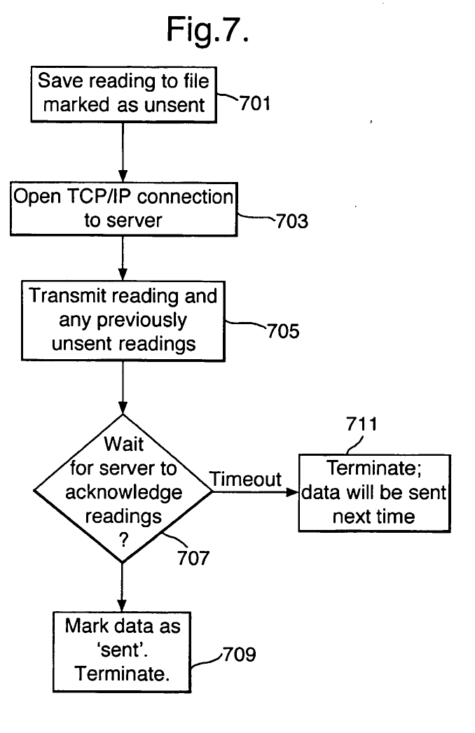

FIG. 7 is a flow diagram of another part of the operation of an embodiment of the invention;

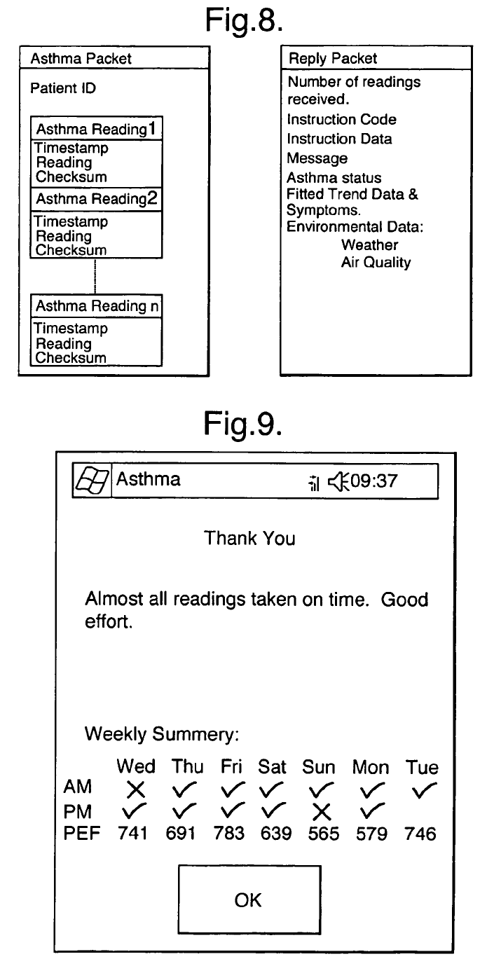

FIG. 8 illustrates the data packet format; and

FIG. 9 illustrates an example of a display to the patient

A first embodiment of the invention as illustrated in FIG. 1 is for use by patients suffering from asthma. The system includes an electronic flow meter 1 which is connected via a cable 3 to a GPRS cellular telephone 5. The cellular telephone 5 is connectable via the GPRS wireless network 7 to a remote server 9. As illustrated in FIG. 1 a clinician such as a general practitioner (GP) 11 may communicate with the server via the internet 13 using a conventional telephone line 15 (another communications link can be used, such as a wireless connection of course) and ISP 17. While a cellular telephone is illustrated and mentioned below, this may be replaced by a PDA with telephone functionality as mentioned above.

GPRS telephones can maintain a permanent connection to the GPRS network whenever they are on. Thus the user does not need to initiate any form of dial-up or connection or session request. In this embodiment the GPRS telephone is provided with a software application which handles the interfacing to the electronic flow meter 1 and the transmission of the data to the remote server 9. The steps required by the patient, together with the automatic operations which are conducted in the background (invisible to the patient) are illustrated in FIG. 2. The first steps 201, 203 are for the patient to connect the GPRS telephone and peak flow meter together using the cable 3 (the cable may be replaced by a Bluetooth or other short range wireless connection) and to switch on the phone and peak flow meter (these steps may be in the other order). As just mentioned, when the GPRS telephone is switched on it automatically establishes a connection to the GPRS network without the intervention of the user as illustrated at 205. The user selects in step 207 an icon on the GPRS telephone to start the software application for taking the measurement. In this embodiment the GPRS telephone is a conventional one which has other functions. However the GPRS functionality may be dedicated to the flow rate meter.

The step of selecting the software application may be eliminated by starting the application automatically on switching on and connection. This may be achieved in one embodiment by providing an intelligent data head 4 on the connection cable 3 which interfaces between the telephone and the medical device. The data head 4 may include a programmable integrated circuit which implements this functionality in conjunction with software on the telephone if necessary.

The operation of the GPRS telephone 5 under control of the software application is illustrated in FIGS. 6 and 7. As illustrated in steps 601 and 602 the telephone starts a child process to read the physiological data from the flow meter 1. In this embodiment the data is made available at an RS-232 port on the peak flow meter 1. Therefore in step 602 the telephone opens the RS-232 port and initialises ready to receive data, for instance by setting time-outs, baud rate etc. At step 209 the telephone then requests that the patient takes the peak flow reading (in fact three times) by displaying the instruction as shown in FIG. 3. It then waits for data as illustrated in step 603 and checks the received data for completeness as illustrated in step 604. Once the data is complete the software formats the data for transmission over the GPRS network by forming it into appropriate data packets which include a patient identifier, a time stamp and the raw data from the peak flow meter. These data packets are automatically transmitted in real time (i.e. immediately upon receipt of data from the peak flow meter) as illustrated in step 605. GPRS once connected allows data to be sent as though on a normal network (e.g. LAN or Ethernet). A TCP/IP socket connection is opened by the software to the server and the data is transmitted in the packet structure illustrated in FIG. 8. The transmission packet for the data, labelled “Asthma Packet” in FIG. 8, includes a patient identifier (ID), and the readings each consisting of a timestamp, the reading and a checksum.

The timestamp provides a degree of authentication and security. To this end the system time can be set by a secure clock which can be conveniently provided in the data head and synchronised to the server by an authenticated communication. Alternatively the secure clock may be provided elsewhere, such as on a specially adapted memory card for the telephone, and it may be with the secure data storage area discussed below. The use of a secure clock is more reliable than relying on the clock in the telephone or device which may easily be reset.

In this context “secure” means that access is given only through authenticated, and optionally encrypted, communication with the server and/or handset software.

The reply packet from the server to the patient indicates the number of readings received (for confirmation purposes), and the additional data which it is desired to send to the patient, which may include Instruction Code, Instruction Data, Message, Asthma Status, Filtered Trend Data & Symptoms and Environmental Data such as Weather and Air Quality.

The data sent to the server can also include an indication of the patient's location. This can be taken from the cell location of the telephone, or from a Global Positioning System (GPS) receiver included in the telephone or device. This opens the possibility of monitoring environmental effects by looking at patients from a defined area.

As illustrated in FIG. 2, the sending of the data to the server as step 210 is invisible to the user and occurs as the user is blowing into the peak flow meter, thus each reading is sent as it is taken. The remote server 9 acknowledges the data it has received at step 212 and on receipt of the acknowledgement the GPRS telephone 5 indicates to the patient that the measurement is satisfactory and that the procedure can be concluded at step 216. In the event of the network connection being unavailable the GPRS telephone stores the data for later transmission as indicated in step 218.

FIG. 7 illustrates in more detail the data transmission process. In step 701 the data is saved to a file marked as unsent. When a connection becomes available in step 703 the connection to the server is opened and the readings (and any previously unsent readings) are sent to the server in step 705. The software waits for an acknowledgement from the server at step 707, and if it receives the acknowledgement the data is marked as sent and the procedure terminated at step 709. However, if no acknowledgement is received within a time-out period then the data is left as unsent and a further attempt is made later as illustrated at 711. The file may be stored in an area of non-volatile memory which provides a secure data storage area. This may be provided in the data head 4 (or correspondingly Bluetooth module in the case of wireless connection), on a SIM or Flash memory card in the telephone or medical device. Modifications, additions or deletions to the data stored on this non-volatile memory can only occur by authenticated, and optionally encrypted, communication with the software on the telephone or the medical device. A log of connections and user interactions is also maintained, this being sent to the server on an automated, and optionally manual, basis.

The software application on the telephone may include some analysis capability at least to detect critical medical conditions so that the patient can be alerted to seek assistance even if the connection to the server is unavailable at that time.

As mentioned above, in this embodiment the data head 4 provided on the cable 3 (or in a Bluetooth module) includes the secure clock, the secure data storage area and a processor for handling the interfacing. This has the advantage that the memory and clock on the telephone/PDA is not particularly critical, and that the functionality related to the medical application is concentrated in the data head 4. Thus where regulatory approval is required for medical devices, regulatory approval of the data head can be obtained, without the need to obtain approval of every type of telephone/PDA that will be used. In other embodiments the secure clock and/or secure memory functionality can be provided separately from the connection, e.g. in a customised memory card.

At the server 9 the data is analysed and may be compared with previous data, e.g. known trends. The comparison can be with data for that patient, and with data for other patients, e.g. a group of patients. The group may be defined by symptoms, geographical area (using the cell locator or GPS data), or other criteria. If the new measurements are within the limits appropriate for the patient, the data is simply added to the patient's file on the server. However, if the readings are identified as causing concern, the server will notify the clinician 11 who can then access the relevant patient data on the server via a secure web page, and can also contact the patient (either by using the GPRS network 7 or in another way). The readings stored on the server will of course be accessed by the clinician during a patient's regular visit to the asthma clinic. In contrast to manually recorded data, the clinician can be sure that the data is reliable and quantitative.

If no measurements have been received at the server for more than a pre-set length of time, such as a day, the server automatically sends a message (e.g. a text message) to the GPRS phone requesting new data from the patient.

As illustrated in FIG. 3 the data collected may also be displayed to the patient. The cellular telephone may also include the provision for the patient to enter comments, for instance to keep an electronic patient diary. This may also be transmitted to the remote server 9 along with the peak flow readings. Where patient input is required appropriate default values (for example based on previous data entry by the patient) are displayed so as to relieve the data entry burden on the patient as much as possible. Other data may also be sent if appropriate, for example images from an imaging device (which may be included in the telephone).

Although only one patient device 1, 3, 5 is illustrated in FIG. 1, it will of course be appreciated that many patients will be provided with the devices, all of whom may be served by the same remote server 9.

From time to time it may be necessary to update the software on the cellular telephone or the medical device. This can conveniently be achieved without user-intervention by automatic download controlled by the server 9. In one embodiment the updating can be triggered according to the patient's condition. For example, if the patient's condition changes it may be that a change of the scripts displayed to the patient is required, such as to ask an additional question which the patient answers by making an entry in the patient diary, or to require a change in the data collection routine. Thus the data displayed to the patient may change depending on the patient's condition as measured by the medical device.

FIG. 4 illustrates twelve weeks worth of data for an example patient using the embodiment of FIG. 1. In the top graph (A) the daily peak flow values are shown by the lighter line, while the trend (explained later) is shown by the heavier line. The second graph (B) indicates use recorded by the patient of the asthma reliever (puffer), and the third graph (C) indicates a subjective measure of the severity of their symptoms as recorded by the patient.

FIG. 9 illustrates an example of a display to the patient of a weekly summary of the readings taken by way of encouragement of diligent recording.

It will be appreciated that the system above is an improvement over requiring manual recording of peak flow readings, and also over previous proposals for telemedicine systems. The operations required by the patient are very simple and quick and do not require any significant familiarity with computer systems, modems or the internet. All that is required is that the equipment is switched on, connected together and the readings taken. The downloading, formatting and transmission of data are entirely invisible to the user.

Although the embodiment above has been described with reference to asthma suffers who need to take peak expiratory flow readings, the system is also applicable to other types of chronic conditions, such as hypertension, diabetes, using appropriate electronic medical devices.

For example, FIG. 5 illustrates a system for monitoring of blood sugar levels for Type I diabetics. This is based on the use of an electronic blood glucose meter 51 of the type which measures blood glucose level in a sample of blood applied by the patient to a test strip 52 inserted into the meter. As before, the blood glucose meter 51 is connected by a RS-232 cable 53 to a GPRS telephone 55 which communicates with a remote server 9 and with a clinician 61 in the same way as the first embodiment of the invention. Thus the patient is required to switch the blood glucose meter on, connect the RS-232 cable 53 to the GPRS telephone 55 and then place a drop of blood on the reagent strip 52 and introduce it into the blood glucose meter. The introduction of the test strip triggers the measurement and the delivery of data to the GPRS telephone 55 which automatically checks, displays, formats and transmits the data to the remote server 9 as before. Again, the remote server can analyse the data and automatically notify any significant departure from expected behaviour to the clinician 61 and possibly to the patient as well. Further, when the patient attends a diabetes clinic, the clinician can access the patient data from the server 9, again in the sure knowledge that the data is reliable and quantitative.

With the system of the invention local information, such as the nearest pharmacy, hospital or clinic may be sent from the server to the patient device. It is also possible for repeat prescriptions of drugs, or other advice relating to the action necessary (eg diet), to be sent in response to the proper monitoring of the condition by the patient taking the readings as scheduled. Medical personnel can be unwilling to give such advice, and certainly unwilling to authorise repeat prescriptions of drugs without examining the patient, which reduces the practical effectiveness of previously proposed telemedicine systems. The problem is overcome with the invention because the advice or prescription follows the secure receipt at the server of measurements of the patient's condition. Thus the system allows self-management of their condition by the patient and the advantages of telemedicine to be obtained.

With any system handling medical data security and confidentiality are important considerations. In the embodiments above the cellular telephone include a digital certificate and the application running on the cellular telephone requires the user to enter a user name and password, and optionally to acquire a biometric such as a fingerprint. The data packets sent to the server are encrypted and digitally signed with the digital certificate. This ensures that the data is authentic and prevents unauthorised software being used to communicate with the server.

As mentioned above these embodiments of the invention include the facility for automatic data analysis at the server 9, for instance to spot trends in the data for individual patients which might require medical intervention. As an example, the server may smooth the data using a scalar Kalman filter, the aim being to spot impending events as they develop (e.g. a significant decrease in peak flow readings in the run-up to a possible “asthma attack”) and to alert the clinician and/or the patient. This form of event detection is tuned to each patient's characteristics and the advice sent to the patient, preferably mediated by the clinician, is to vary the medication and/or its dosage. In FIG. 4, the trend calculated by means of a Kalman smoother is illustrated in the solid line. The Kalman filter is a generic framework for analysis of a linear dynamical system (in this case, the time-dependent peak flow, blood glucose or blood pressure readings). Using a process model, the next state x is computed from the current state using a transition matrix A and assuming first-order (Markov) dynamics with process noise Q i.e. X(t+1)=AX(t)+Q. The observation model relates the measurements Y to the state of the system via the observation matrix C and observation noise R, ie. Y(t)=CX(t)+R. The process and observation noise Q and R are assumed to be independent and to have zero mean. The peak flow values (or blood glucose levels or blood pressure measurements) can be modelled with a scalar Kalman filter which assumes that the next value will be the same as the current value (this means that A is equal to 1) plus some process noise characterising normal variability. In addition, it is also assumed that C=1, i.e. the peak flow value (or blood glucose level or blood pressure measurement) is both the measurement Y and the state X of the system. In this instance the scalar Kalman filter is run as a Kalman smoother of the raw data, which, with suitable values for the process and measurement noise, allows the filter to perform on-line trend analysis of a noisy or oscillatory set of readings as shown in the above plot. In the plot in FIG. 4 the process noise Q was taken as 10 and the observation noise R as 100 with initial values of the state X as 300 and of the state variance V as 40. Thus, the trend in FIG. 4, shown as a heavy line in graph (A), is not affected by the highly oscillatory, nature of the readings in the early part of the period(early April), and correctly identifies the clinically significant dip in peak flow values later (in mid-May), which coincides with increased use of the reliever by the patient (B) and a more severe self-assessment of symptoms (C).

The use of the above system is not only beneficial to the patient in reducing the time and trouble needed for self-monitoring, but also manifestly improves the reliability of the data itself. Also, with conventional systems self-monitoring by patients just occurs independently, in the field, and is only reviewed at regular clinics. With this system the clinician is always available in the patient management process loop. This means that the patient's condition can be monitored and controlled more effectively—in near real time, which in turn reduces the likelihood of long-term complications and reduces the need for emergency or extreme measures caused when the patient's condition has departed too far from an acceptable stable state. Such changes in condition can be identified sooner, particularly with the automatic trend analysis at the server, rather than only when the patient's condition becomes critical or only when the patient visits the clinic. It therefore reduces the need for serious medical intervention which is of benefit both to the patient and to the medical services.

With the systems described above, the fact that the monitoring can be virtually guaranteed to be accurate (because of the automatic transmission of the raw data), regular (because of the ease of the procedure and the availability of reminders from the server), and can spot dangerous trends means that the frequency of clinic visits could be reduced. This is therefore more convenient for the patient and cost-effective for the medical services.

1. A telemedicine system comprising a patient-based physiological data acquisition and transmittal device connectable via a wireless network to transmit physiological data to a remote server, wherein the patient-based measurement and data transmittal device comprises:
an electronic physiological data acquisition unit for measuring a physiological parameter of a patient to acquire and output data representing the parameter;
a wireless transmitter which upon receiving the output data from the data acquisition unit automatically transmits the output data via the wireless network to the remote server.

2. A telemedicine system according to claim 1 wherein the wireless transmitter is adapted to receive automatically the output data from the physiological data acquisition unit on data acquisition thereby, and thereupon automatically to transmit the output data immediately in real time to the remote server.

3. A telemedicine system according to claim 1 wherein the wireless transmitter is adapted to establish a connection to the wireless network automatically when it is switched on and to maintain the connection while switched on.

4. A telemedicine system according to claim 1, wherein the wireless network is a packet-switched network.

5. A telemedicine system according to claim 4 wherein the wireless network is a public network.

6. A telemedicine system according to claim 5 wherein the wireless network is the General Packet Radio Service (GPRS) network.

7. A telemedicine system according to claim 1, the wireless network is the 3G, PDC-P or EDGE network.

8. A telemedicine system according to claim 1 wherein the wireless transmitter is a cellular telephone/pda.

9. A telemedicine system according to claim 8 wherein a software application is provided on the cellular telephone/pda to interface with the physiological data acquisition unit and to control data transmission to the remote server.

10. A telemedicine system according to claim 1 wherein the patient-based measurement and data transmittal device is adapted to check the acquired data for compliance with preset conditions.

11. A telemedicine system according to claim 10 wherein the preset conditions relate to the quality or completeness of the data or the condition of the patient.

12. A telemedicine system according to claim 1 wherein the patient-based measurement and data transmittal device comprises a display for displaying the data to the patient.

13. A telemedicine system according to claim 1 wherein the patient-based measurement and data transmittal device stores the data if a network connection is unavailable and automatically retransmits it later when a network connection is available.

14. A telemedicine system according to claim 1 wherein the remote server processes the data to check the condition of the patient and responds with a message via the wireless network.

15. A telemedicine system according to claim 1 wherein the remote server formats the data for delivery and display to a clinician.

16. A telemedicine system according to claim 1 wherein the remote server comprises a data analyser for identifying trends in the data and a message generator for generating messages to be output to at least one of the patient and a clinician.

17. A telemedicine system according to claim 16 wherein the data analyser comprises a Kalman smoother for smoothing the data.

18. A telemedicine system according to claim 1 wherein the physiological data acquisition unit is one of: an electronic flow meter for recording Peak Expiratory Flowrate, an electronic blood glucose meter, a blood pressure monitor, and a heart rate monitor.

19. A telemedicine system according to claim 1 wherein the physiological data acquisition unit and wireless transmitter are integrated as a single device.

20. A telemedicine system according to claim 1 wherein the data sent from the wireless transmitter is time stamped with reference to a secure clock.

21. A telemedicine system according to claim 20 wherein the secure clock is provided in the patient-based physiological data acquisition and transmittal device.

22. A telemedicine system according to claim 1 wherein a secure data store is provided in the patient-based physiological data acquisition and transmittal device.

23. A telemedicine system according to claim 1 wherein the data sent from the wireless transmitter is digitally signed.

24. A telemedicine system according to claim 1 wherein the data sent from the wireless transmitter comprises the location of the wireless transmitter.

25. A telemedicine system according to claim 24 wherein information is sent from the server to the patient-based physiological data acquisition and transmittal device for display thereon and is adapted depending on the location of the wireless transmitter.

26. A telemedicine system according to claim 1 wherein information is sent from the server to the patient-based physiological data acquisition and transmittal device for display thereon to initiate interaction with the patient and is adapted depending on the value of the physiological parameter measured by the electronic physiological data acquisition unit.

27. A telemedicine system according to claim 1 wherein information is sent from the server to the patient-based physiological data acquisition and transmittal device, and wherein in dependence upon said physiological parameter measurement and transmission to the server said information comprises a prescription for medication.

28. A telemedicine system according to claim 1 wherein the electronic physiological data acquisition unit is connectable to the a wireless transmitter by a connection comprising a data head including an interface.

29. A telemedicine system according to claim 28 wherein the data head comprises a secure clock for time stamping the data.

30. A telemedicine system according to claim 28 wherein the data head comprises a secure memory for storing the data.

31. A telemedicine system which incorporates handset delivery of advice relating to changes in medication necessary to control a respiratory condition including asthma.

32. A telemedicine system according to claim 31 wherein the handset comprises a graphical device indicating the state of an asthmatic condition relative to an alert level.

33. A telemedicine system according to claim 31 wherein the medication advice is based on readings analysed by software at the server and/or handset.

34. A telemedicine system which incorporates handset delivery of geographically local information relevant to the patient condition from a central server, such information being derived from knowledge of the geographic location of the wireless handset and being adapted based on measurement of the patient condition by the telemedicine system.

35. A telemedicine system according to claim 34 wherein said local information comprises local air quality information and weather conditions relevant to patients with respiratory diseases.
